(19) United States
(12) Patent Application Publication
(10) Pub. No.: US 2003/0072824 A1
(43) Pub. Date: Apr. 17, 2003

(54) Ume extract having medicinal effects and compositions containing the same

(76) Inventors: Masayuki Yoshikawa, Osaka (JP); Teruaki Hayashi, Hyogo (JP); Yoshihiko Higashi, Wakayama (JP)

Correspondence Address: MERCHANT & GOULD PC, P.O. BOX 2903, MINNEAPOLIS, MN, 55402-0903, US

(21) Appl. No.: 09/868,710

(22) PCT Filed: Dec. 24, 1999
(86) PCT No.: PCT/JP99/07285

(30) Foreign Application Priority Data
Dec. 25, 1998 (JP) 10/371331
Aug. 25, 1999 (JP) 11/238476

(51) Int. Cl. A61K 35/78

(52) U.S. Cl. 424/769; 424/725

(57) ABSTRACT

Extracts are prepared from a leaf and a stem of ume, a kernel of ume, and an ume flower, using methanol in an amount of 5 times the volume thereof. These extracts have anti-oxidation action, gastric mucosa injury inhibiting action, aldose reductase inhibiting action, blood glucose level elevation inhibiting action, platelet agglutination accelerating action, alcohol absorption inhibiting action, anti-inflammatory action, and the like.

### DESCRIPTION

1. Technical Field

The present invention relates to ume (Japanese apricot: Prunus mume) extracts having medicinal effects and compositions containing the extracts.

2. Background Art

Traditionally, many ume trees are raised to produce edible ume. In the present state, however, parts of ume tree other than ume flesh or unripe ume extract are disposed of as industrial waste. In raising ume trees, for example, abnormally long branches are pruned every autumn, and this amounts to thousands of tons to tens of thousand of tons per year. The pruned branches are burned as waste. In addition, most seeds and shells after drawing unripe ume extract or ume flesh also are discarded. This is not desirable in view of effective use of resources and environmental conservation.

On the other hand, in the field of medicine and pharmacy, medicines derived from organisms in nature (including what are called herb medicine and crude drug) are drawing greater attention than chemically synthesized medicines. This is because even if synthetic medicines are excellent in their efficacy, they have problems of side effects. On the other hand, substances separated from organisms existing in nature, particularly organisms that have been eaten by humans from ancient times, are considered to be excellent in safety. Furthermore, there are a variety of organisms depending on their living environment and the like, and substances separated from them have unknown possibilities as medicines. Ume is particularly expected to have such uses, since it has been considered to be good for human health from ancient times, and also is considered to be holy, and is used in religious ceremonies. If a medicine can be developed from ume for any disease that is currently a significant problem, it also can make social contribution.

Thus, it is an object of the present invention to provide an ume extract having a medicinal effect, and compositions containing the extract, which can contribute to the effective use of ume.

### DISCLOSURE OF THE INVENTION

To accomplish the above object, the inventors have carried out a series of researches on the medicinal effects of extracts from various parts of ume. As a result, ume extracts having an excellent medicinal effect were able to be obtained, as shown in the following.

That is, the present invention provides a first ume extract having a medicinal effect, the first ume extract being extracted from at least one selected from the group consisting of a main stem of ume tree, a branch of ume tree, a leaf of ume tree, a stem of ume tree, a root of ume tree, ume flesh, seed and shell of ume, and a kernel of ume, wherein the first ume extract is used as any of an antioxidant, a gastric mucosa injury inhibitor, a diabetic cataract preventing agent with aldose reductase inhibition activity, a diabetic neurosis preventing agent with aldose reductase inhibition activity, a blood glucose level elevation inhibitor, an alcohol absorption inhibitor, a platelet agglutination accelerator, a hepatitis inhibitor, an anti-inflammatory agent, and a melanin pigment formation inhibitor with tyrosinase inhibition activity.

Among these, an extract from at least one of a leaf of ume tree and a stem of ume tree is very excellent and is preferred as an antioxidant, a diabetic cataract preventing agent, a diabetic neuronosis preventing agent, a platelet agglutination accelerator, an anti-inflammatory agent or a melanin pigment formation inhibitor. A stem of ume tree herein refers to a part present between a leaf and a branch for supporting the leaf. Hereinafter, a leaf and a stem are together called a leaf-stem.

When the first ume extract is used as a gastric mucosa injury inhibitor or an alcohol absorption inhibitor, preferably it is extracted from a kernel of ume. This is because such an extract is excellent in those medicinal effects.

When the first ume extract is used as a blood glucose level elevation inhibitor or a hepatitis inhibitor, the extract exhibits an excellent medicinal effect when extracted from either of a kernel and a leaf-stem of ume.

It is preferable that the first extract contains at least one substance selected from the group consisting of six substances represented by the following chemical formulae (1) to (6). This is because these substances are presumed to be involved in the above-mentioned various medicinal effects.

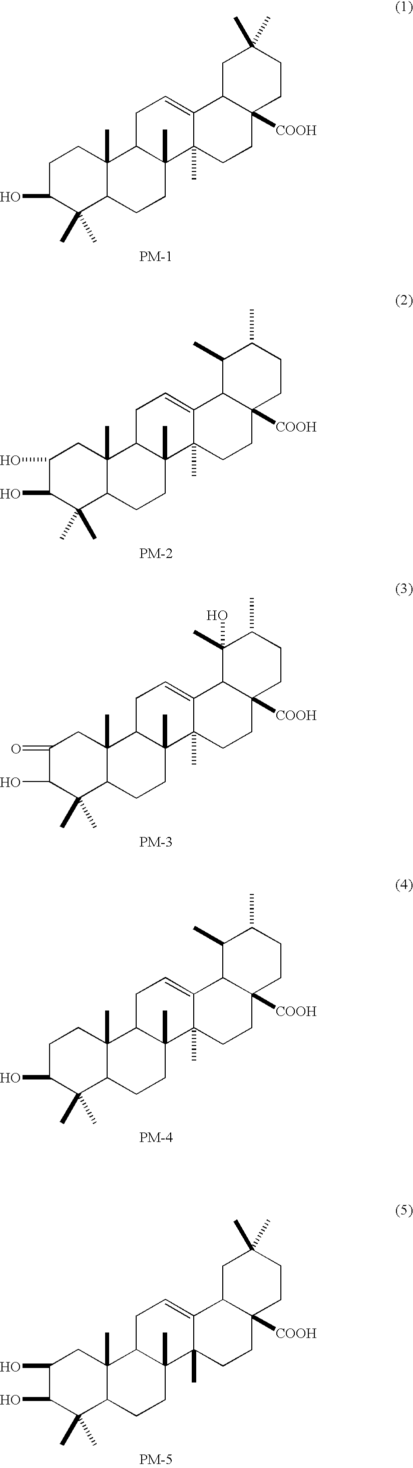

In the following, the substances of the formulae (1), (2), (3), (4), (5) and (6) are referred to as PM-1, PM-2, PM-3, PM-4, PM-5 and PM-6, respectively. The names of these substances are as follows:

PM-1: 3β-hydroxy-12-olean-28-oic acid

PM-2: 2α,3β-dihydroxy-12-ursen-28-oic acid

PM-3: 3β,19α-dihydroxy-2-oxo-12-ursen-28-oic acid

PM-4: 3β-hydroxy-12-ursen-28-oic acid

PM-5: 2α,3β-dihydroxy-12-olean-28-oic acid

PM-6: 2α,3α-dihydroxy-12-olean-28-oic acid

Next, the present invention provides a second extract, the second extract being extracted from an ume flower, wherein the second extract is used as any of a diabetic cataract preventing agent with aldose reductase inhibition activity, a diabetic neurosis preventing agent with aldose reductase inhibition activity, an anti-inflammatory agent, an antioxidant, a melanin pigment formation inhibitor with tyrosinase inhibition activity and a platelet agglutination inhibitor. In the present invention, an ume flower refers to a reproductive organ of ume consisting of pistil, stamen, petal, sepal, peduncle, bract and the like.

It is preferable that the second extract contains at least one substance selected from the group consisting of eight substances represented by the following chemical formulae (7) to (14). This is because these substances are presumed to be involved in the above-mentioned various medicinal effects.

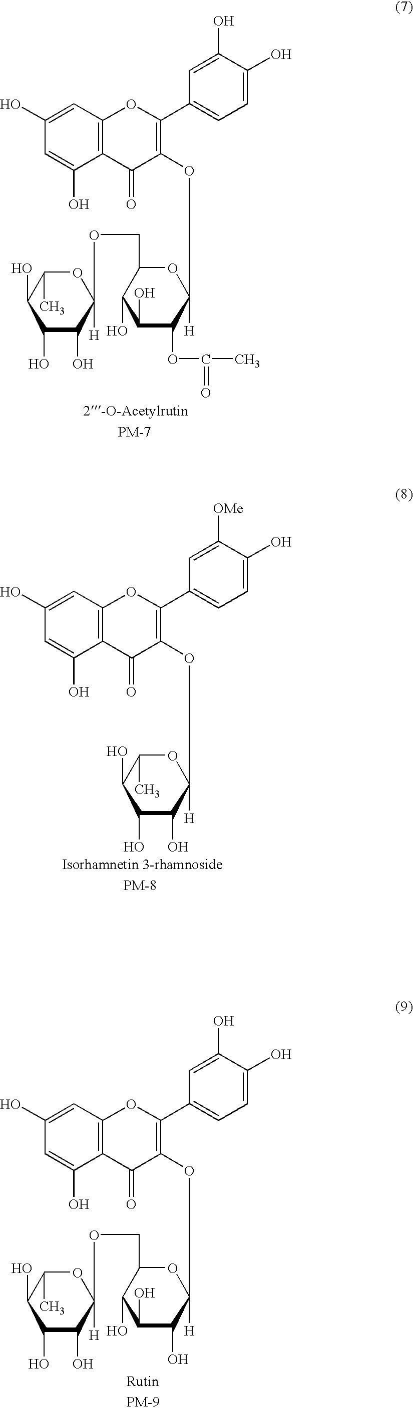

In the following, the substances of the formulae (7), (8), (9), (10), (11), (12), (13) and (14) are referred to as PM-7, PM-8, PM-9, PM-10, PM-11, PM-12, PM-13 and PM-14, respectively. The names of these substances are as follows:

PM-7: 2′″-O-acetylrutin

PM-8: isorhamnetin 3-rhamnoside

PM-9: rutin

PM-10: quercetin 3-O-rhamnopyranosyl(1→6)galactoside

PM-11: quercetin 3-O-neohesperidoside

PM-12: eugenylglucoside

PM-13: benzyl glucopyranoside

PM-14: benzyl alcohol xylosyl(1→6)glucoside

In the present invention, it is preferable that the ume extract is an organic solvent extract, particularly an alcohol extract. As the alcohol extract, at least either one of an ethanol extract and a methanol extract is preferred, and a methanol extract is particularly preferred. Alternatively, it is preferable that the ume extract is a dry distillation extract.

In the present invention, the form of the ume extract is not particularly limited. For example, it may be powdery, or may be in liquid form (including paste form).

Next, the present invention provides a composition containing the ume extract of the present invention. When this composition contains the ume extract of the present invention as a main ingredient or an active ingredient, other ingredients of the composition are not particularly limited, and they are determined as appropriate depending on the use of the composition. For example, when the composition is a medicine, it may contain the ume extract as a main ingredient or an active ingredient, and comprises an excipient in solid or liquid form. When the composition is an internal agent, it may be usually in the form of powder, tablet, capsule, tea, granule or solution (spirit, tincture, fluid extract, syrup or the like). It also may be in the form of injection, ointment, solution, poultice, crude drug, spray, nutrient enema, emulsion or the like. As the excipient, any of those known in the art may be employed. Examples of the excipient of an internal powder in the form of powder, granule, capsule or tablet include lactose, starch, dextrin, calcium phosphate, calcium carbonate, synthetic or natural aluminum silicate, magnesium oxide, dry aluminum hydroxide, magnesium stearate, sodium bicarbonate, dry yeast, and the like. Examples of the excipient of an external powder include zinc oxide, talc, starch, kaolin, boric acid powder, zinc stearate, magnesium stearate, bismuth subgallate, aluminum potassium sulfate powder, and the like. Examples of the excipient of the solution include water, glycerol, propylene glycol, syrup, ethanol, fatty acids, ethylene glycol, polyethylene glycol, sorbitol, and the like. Examples of the excipient of an ointment include hydrophobic bases or hydrophilic bases (including emulsion bases, water-soluble bases and suspension bases) which are prepared by combining fats, fatty oils, lanolin, vaseline, glycerol, cera, Japan wax, paraffin, fluid paraffin resin, higher alcohols, plastics, glycols, water, surfactants, and the like.

When the composition of the present invention is used as a health food or a food additive, it may be used in the form of a tablet, capsule, granule, powder, solution or the like. However, it is used preferably in a form that can be clearly distinguished from a drug. As the excipient of this case, those described above may be employed.

In the composition of the present invention, the content of the ume extract is, for example, from 1 to 60 wt %, preferably from 10 to 40 wt % with respect to the total composition.

### BRIEF DESCRIPTION OF THE DRAWINGS

FIG. 1 is a chart in which platelet agglutination acceleration action of an ume extract was confirmed in one example of the present invention;

FIG. 2 is a diagram showing a purification process of a medicinal ingredient in an ume extract in another example of the present invention;

FIG. 3 is a chart in which an infrared absorption spectrum of an ume extract was measured in still another example of the present invention;

FIG. 4 is a chart in which an infrared absorption spectrum of an ume extract was measured in still another example of the present invention;

FIG. 5 is a chart in which platelet agglutination acceleration action of an ume extract was confirmed in still another example of the present invention;

FIG. 6 is a chart in which platelet agglutination acceleration action of an ume extract was confirmed in still another example of the present invention;

FIG. 7 is a chart in which platelet agglutination acceleration action of an ume extract was confirmed in still another example of the present invention; and

FIG. 8 is a diagram showing a purification process of a medicinal ingredient in an ume extract in still another example of the present invention.

### BEST MODE FOR CARRYING OUT THE INVENTION

An ume extract of the present invention can be produced, for example, as follows:

First, when extracting with an organic solvent, a branch, a leaf-stem or the like of an ume tree is machined into flakes. This machining can be effected using a cutter for wood or the like for hard parts of ume tree such as a main stem, a branch and a root, and using a cutter for food, a mixer, or the like for soft parts such as ume flower and a leaf-stem.

Then, flakes of a branch or the like of the ume tree are immersed into an organic solvent to carry out extraction. Examples of the organic solvent include methanol, ethanol, hexane, acetone, ethyl acetate, glycerol, propylene glycol, n-butanol and the like. Among these, as described above, methanol or ethanol is preferred, and methanol is particularly preferred. These organic solvents may be used either alone or in combinations of two or more kinds. The amount of the organic solvent used is usually from 5 to 10 times the volume of the flakes, preferably about 5 times the volume of the flakes. The immersion (extraction) may be carried out one time, however, it is preferably carried out at least two times. Preferably, extraction is effected by heating reflux. In this case, the time required for the extraction is usually from 1 to 3 hours, preferably about 3 hours. The temperature of the organic solvent is determined based on the type of the solvent as appropriate, however, a temperature at which reflux of the extraction solvent takes place, specifically from about 50° C. to about 80° C., is preferred. Furthermore, when extraction is carried out at room temperature, the extraction time is preferably one whole day and night. According to this extraction, a medicinal ingredient is extracted in an organic solvent from a branch or a leaf-stem of ume tree, an ume flower, or the like. Then, flakes of the branch or the like of an ume tree are separated with a filter, etc., and an extract is recovered.

The extract may be used as it is, or may be formed into powder or paste by evaporating (e.g. drying under reduced pressure) the organic solvent.

Next, when extraction is carried out by dry distillation, first, in the same manner as the above, a branch, a leaf-stem, or the like of ume tree is machined into flakes. Then, these flakes are heated while cutting off air (dry distillation). The heating temperature at this time is usually from 90° C. to 300° C., and the heating time is usually from 1 to 3 hours. In this dry distillation, for example, a retort furnace or a dry distillation furnace may be used. A liquid that is allowed to flow through this dry distillation is separated from the branch or the like, and recovered as an extract. This dry distillation extract may be used as it is, or may be formed into powder or paste by drying.

Although the kind of the ume used in the present invention is not particularly limited, for example, nankoume, kotsubunanko, gojiro, kafryouchida, shirakaga, yosei, rinsyu, osyuku, kosyukoume, benisashi, kaidare and the like may be employed.

In the following, examples of the present invention are described. The ume supplied in the following examples is nankoume.

### EXAMPLE 1

In this Example, gastric mucosa injury inhibition action of methanol extracts of ume kernel and ume leaf-stem were confirmed.

Preparation of Leaf-Stem Extract

5.1 kg of finely ground leaf-stem of ume was heat refluxed for 3 hours with methanol in a volume of five times that of the leaf-stem to carry out extraction. The resultant extract was filtered, and thereafter the residue was heat refluxed again with methanol in the same manner as the above to carry out extraction. The obtained extracts were joined, and dried under reduced pressure to obtain 338 g of an extract. The properties of this extract are shown below.

Appearances and Properties

Dark brown, paste form, with a trace of peculiar odor.

Thin Layer Chromatography

(1) Conditions

Carrier: Silica gel (60F254, manufactured by Merck & Co., Inc.)

Developing solvent: Mixture of chloroform: methanol: water=10:3:1 (volume ratio)

Coloration: Heating with 10% cerium sulfate and 10% sulfuric acid aqueous solution

(2) Rf value

Spot 1: 0.70 (red)

Spot 2: 0.63 (brown)

Infrared Absorption Spectrum

(1) Measuring apparatus: Shimadzu FT-TR DR-8000 spectrometer

(2) Measurement result: (unit: cm⁻¹)

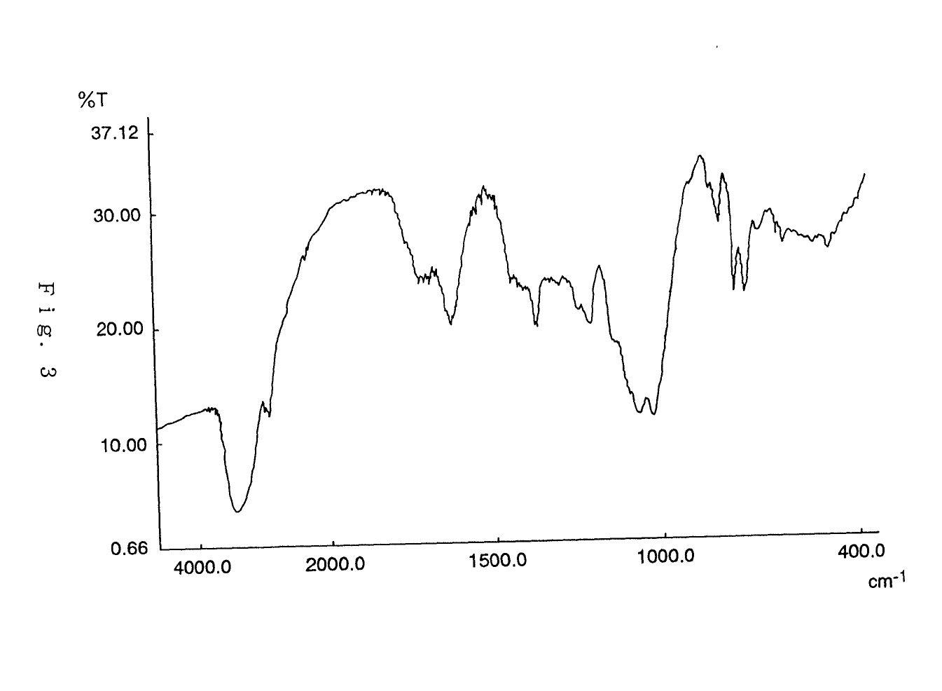

The extract had characteristic absorptions at 3432 (hydroxyl group), 2936 (methyl group, methylene group, methane group), 1736 (carbonyl group), 1655 (unsaturated bond), 1383 (methyl group, methylene group, methane group), 1109, 1082, 1037 (hydroxyl group, ether bond), 792, and 760 (unsaturated bond). This result is shown in the chart of FIG. 3.

Preparation of Ume Kernel Extract

2.5 kg of finely ground ume kernel was heat refluxed for 3 hours with methanol in a volume of five times that of the ume kernel to carry out extraction. The resultant extract was filtered, and thereafter the residue was heat refluxed again with methanol in the same manner as the above to carry out extraction. The obtained extracts were joined, and dried under reduced pressure to obtain 223 g of an extract. The properties of this extract are shown below.

Appearances and Properties

Dark brown, paste form, with a trace of peculiar odor.

Thin Layer Chromatography

(1) Conditions are the same as the above

(2) Rf value

Spot 1: 0.10 (brown)

Spot 2: 0.95 (brown)

Infrared Absorption Spectrum

(1) Measuring apparatus: the same apparatus as the above

(2) Measurement result: (unit: cm⁻¹)

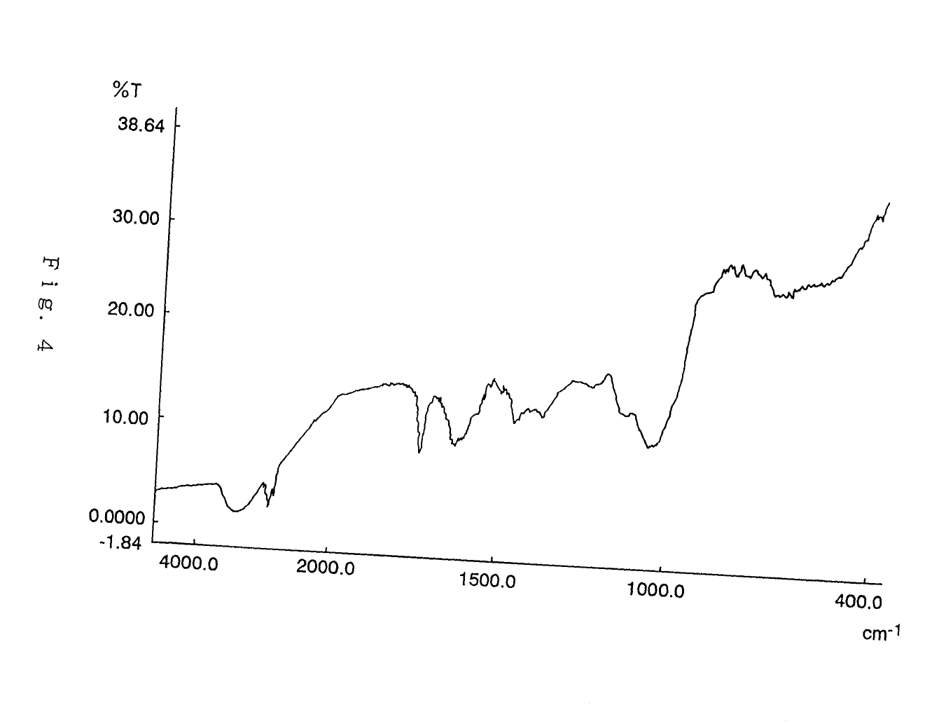

The extract had characteristic absorptions at 3453 (hydroxyl group), 2928 (methyl group, methylene group, methane group), 2500 to 2000 (phenolic hydroxyl group, carboxyl group), 1746 (carbonyl group), 1655 (unsaturated bond), 1072, and 1051 (hydroxyl group, ether bond). This result is shown in the chart of FIG. 4.

Confirmation of Action of Inhibiting Gastric Mucosa Injury

SD male rats (about 250 g in weight) were fasted for about 24 hours, and thereafter the above-mentioned methanol extract was orally administered. Then after one hour, methanol was orally administered in an amount of 1.5 ml per rat. Then after another one hour, the stomach was extracted from each rat. The extracted stomach was treated with formalin, and the maximum diameter of the injuries generated in the glandular stomach region (injury coefficient (mm)), and the score (0 to 9) based on the standard below were determined. As a control, the injury coefficient (mm) and the score (0 to 9) also were determined with respect to rats to which the methanol extract was not administered. An inhibition ratio (%) was determined according to the equation below. These results are shown in Table 1 below. In this table, the values are indicated by average value±standard error (significant difference: * P<0.05, ** P<0.01). This convention is also used in other tables.

Inhibition ratio (%)=100−[(C/A)×100]

A: Injury coefficient of rats to which the methanol extract was administered.

C: Injury coefficient of control rats

Score

0: No injury

1: Less than five small injuries with an overall length of not more than 5 mm and a width of not more than 2 mm

2: At least five small injuries with an overall length of not more than 5 mm and a width of not more than 2 mm

3: Less than five medium injuries with an overall length of at least 5 mm and a width of not more than 2 mm

4: At least five medium injuries with an overall length of at least 5 mm and a width of not more than 2 mm

5: One to three hemorrhagic bands as medium injuries with an overall length of not more than 5 mm and a width of at least 2 mm

6: At least four hemorrhagic bands as medium injuries with an overall length of not more than 5 mm and a width of at least 2 mm

7: One to three hemorrhagic bands as severe injuries with an overall length of at least 5 mm and a width of at least 2 mm

8: Four to six hemorrhagic bands as severe injuries with an overall length of at least 5 mm and a width of at least 2 mm

9: At least seven hemorrhagic bands as severe injuries with an overall length of at least 5 mm and a width of at least 2 mm

TABLE 1

| | | | | Inhi- | |
|---|---|---|---|---|---|
| | Dose | N | Injury | bition | |
| Type of | (mg/ | (number | coefficient | ratio | |
| ume extract | kg) | of rats) | (mm) | (%) | Score |
| Control | — | 8 | 173.4 ± 16.9 | — | 7.8 ± 0.2 |
| Ume leaf-stem | 250 | 6 | 92.8 ± 22.8** | 46.5 | 5.7 ± 0.7* |
| Ume leaf-stem | 500 | 6 | 24.0 ± 12.4** | 86.2 | 2.2 ± 0.7** |
| Ume kernel | 250 | 6 | 46.1 ± 4.4** | 73.4 | 3.5 ± 0.8** |
| Ume kernel | 500 | 6 | 25.8 ± 11.7** | 85.1 | 1.7 ± 0.7** |

(*P < 0.05, **P < 0.01)

As is apparent from the Table 1, gastric mucosa injury was inhibited by administering the ume methanol extract. The ume kernel exhibited more excellent action than the ume leaf-stem.

### EXAMPLE 2

In this example, anti-oxidation action of a methanol extract of ume leaf-stem was confirmed. The methanol extract of leaf-stem was prepared in the same manner as in Example 1.

Method of Confirming Anti-oxidation Action

An ethanol solution of a stable radical 1,1-diphenyl-2-picryl-hydrazyl (DPPH) has an absorption at 517 nm in blue. When a radical trapping substance is added to this solution, the solution is discolored depending on the amount of the trapping substance added. This phenomenon was utilized in this method. First, 1 ml of ethanol, 1 ml of an ethanol solution of the extract (concentration: 0 to 100 μg/ml), 2 ml of 0.2 M acetic acid buffer solution (pH 5.5) and 1 ml of DPPH solution (2.0×10⁻⁷ mol/ml ethanol solution) were mixed, and the mixture was allowed to stand for 30 minutes. Then, the absorbance of this mixed solution was determined at 517 nm. On the other hand, a mixture solution substituting 1 ml of ethanol for the DPPH solution was used as a blank, and the absorbance of this mixture solution was determined at 517 nm in the same manner as the above. Then, the amount of the extract required to reduce the absorbance to half was calculated, and this was used as an indicator of anti-oxidation. As comparative examples, α-tocopherol and green tea were used in place of the extract, and the anti-oxidation power thereof was investigated. These results are shown in Table 2 below.

TABLE 2

| Sample | Anti-oxidation power |
|---|---|
| Extract of ume leaf-stem | 10 μg |
| α-tocopherol | 21 μg |
| Green tea | 20 μg |

(Anti-oxidation power: the amount required to trap 1.0 × 10⁻⁷ mol DPPH)

As is apparent from the Table 2, the extract of ume leaf-stem had an excellent anti-oxidation power, which is at least twice that of α-tocopherol used as an antioxidant in food and the like.

### EXAMPLE 3

In this example, action of inhibiting blood glucose level elevation of ume kernel and ume leaf-stem was confirmed. In this example, methanol extracts of ume kernel and ume leaf-stem were prepared in the same manner as in Example 1.

Confirmation of Action of Inhihiting Blood Glucose Level Elevation

The above-mentioned extract (500 mg/kg) was orally administered to Wistar male rats (150 to 180 g in weight) that had been fasted for about 20 hours. Then, after 30 minutes, sucrose (1.0 g/kg) was orally administered to the rats. Then, after 30 minutes, one hour, and two hours respectively, about 0.2 ml of blood was drawn from the orbital vein. The blood was centrifuged at 3000 rpm to obtain a serum. The blood glucose level of the serum was measured by a glucose oxidase method using a commercial kit (GLUCOSE CII TEST WACO, manufactured by Wako Pure Chemical Industries, Ltd.). As a normal group, the blood glucose level of the rats to which the extract and sucrose were not administered was measured in the same manner. As a control group, the blood glucose level of the rats to which the extract was not administered and only sucrose was administered was measured. These results are shown in Table 3 below. Six rats were treated in each group.

TABLE 3

| | Blood glucose level (mg/100 ml) | | |
|---|---|---|---|
| Treated group | 0.5 h | 1.0 h | 2.0 h |
| Normal group | 73.1 ± 4.6** | 81.9 ± 6.8** | 80.2 ± 5.3** |
| Control group | 182.0 ± 5.7 | 163.1 ± 4.5 | 109.4 ± 3.5 |
| Ume kernel | 172.7 ± 4.8 | 155.8 ± 9.5 | 107.8 ± 4.4 |
| Ume leaf-stem | 174.5 ± 9.7 | 157.5 ± 4.5 | 109.8 ± 4.6 |

As is apparent from the Table 3, elevation of blood sugar level was inhibited by administering the extracts of ume kernel and ume leaf-stem. With respect to this effect, it appeared that there was not a large difference between a kernel and a leaf-stem.

### EXAMPLE 4

In this example, action of inhibiting alcohol absorption of methanol extracts of ume kernel and ume leaf-stem were confirmed. The methanol extracts were prepared in the same manner as in Example 1.

Confirmation of Action of Inhibiting Alcohol Absorption

The above-mentioned extract (500 mg/kg) was administered orally to Wistar male rats (about 210 to 240 g in weight) that had been fasted for about 20 hours, and then after 30 minutes, ethanol (20% (v/v), 5 ml/kg) was administered orally to the rats. Then after 30 minutes, one hour, and two hours respectively, about 0.5 ml of blood was drawn from the orbital vein. The ethanol concentration in the blood was measured by an enzyme method (using a blood alcohol test “BMY”, which is a commercial kit manufactured by Boehringer Mannheim Co., Ltd.). As a control group, the ethanol concentration in the blood of rats to which the extract was not administered but ethanol was administered was measured in the same manner. These results are shown in Table 4 below. Five rats were treated in each group.

TABLE 4

| | Blood ethanol concentration (mg/ml) | | |
|---|---|---|---|
| Treated group | 0.5 h | 1.0 h | 2.0 h |
| Control group | 0.825 ± 0.051 | 0.671 ± 0.022 | 0.337 ± 0.003 |
| Ume kernel | 0.571 ± 0.154 | 0.586 ± 0.079 | 0.332 ± 0.029 |
| Ume leaf-stem | 0.783 ± 0.147 | 0.760 ± 0.086 | 0.512 ± 0.067 |

As is apparent from the Table 4, alcohol absorption was inhibited by administering the methanol extracts of ume kernel and ume leaf-stem. The action of the ume kernel extract was more excellent than that of the ume leaf-stem extract.

### EXAMPLE 5

In this example, rat lens-derived aldose reductase inhibition activity of methanol extracts of ume kernel and ume leaf-stem was confirmed. Aldose reductase is involved in diabetic cataract and diabetic neurosis. The methanol extracts were prepared in the same manner as in Example 1.

Preparation of Aldose Reductase Solution

5 g of lens of Wistar male rats (6-week old) was homogenized in 20 ml of a phosphate buffer solution (135 mM, pH 7.0, containing 10 mM mercaptoethanol), and centrifuged at 100,000×g for 30 minutes. The resultant supernatant was used as an enzyme solution.

Confirmation of Aldose Reductase Inhihition Activity

135 mM phosphate buffer solution (pH 7.0) containing 1 mM DL-glyceraldehyde, 0.03 mM NADPH, 0.1 M lithium sulfate, the rat lens-derived aldose reductase and a DMSO solution (concentration: 0 to 30 μg/ml) of the extract was incubated at 30° C. for 30 minutes. The reaction was initiated by adding NADPH, and quenched by adding hydrochloric acid. After treating the quenched reaction solution with a strong alkali, it was heated at 60° C. for 10 minutes, and allowed to stand until it reached room temperature. Thereafter, the intensity of the fluorescence of the reaction solution was measured (excitation wavelength: 360 nm, radiation wavelength: 460 nm). The aldose reductase inhibition activity was evaluated as the amount of the extract with which the activity of the aldose reductase is reduced to 50% (IC₅₀ (μg/ml)).

As a result, the ume leaf-stem extract had an lC₅₀ of 4.96 μg/ml, and exhibited an inhibition activity in a very small amount. The ume kernel extract exhibited an inhibition activity of 29.3% at a concentration of 30 μg/ml.

### EXAMPLE 6

In this example, platelet agglutination acceleration action of a methanol extract of ume leaf-stem was confirmed. The methanol extract of ume leaf-stem was prepared in the same manner as in Example 1.

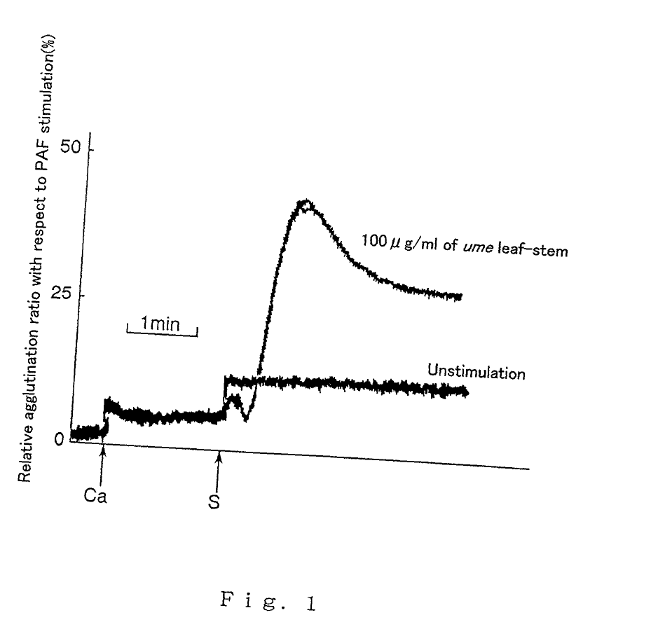

First, washed platelets (5×10⁵ cells/ml) were prepared from whole blood of a Japan white male rabbit by a conventional method. The washed platelets were stimulated with a methanol extract of ume leaf-stem (100 μg/ml) at 37° C. in the presence of 1 ml of Ca²⁺, and the change in the light transmittance at this time was measured (aggregometer: Model PAT-4A, manufactured by Nikko Bioscience Co., Ltd.). As a positive control, the washed platelet were stimulated using a platelet activation factor (PAF) in place of the extract, and the change in light transmittance was measured in the same manner. Considering the agglutination ratio of the positive control as 100%, the platelet agglutination acceleration action of the methanol extract of ume leaf-stem was expressed by a relative ratio with respect to the positive control. This result is shown in the chart of FIG. 1. In this chart, Ca indicates the point when Ca²⁺ was added, and S indicates the point when the agglutination stimulation was initiated.

As is apparent from FIG. 1, when the methanol extract of ume leaf-stem was added, platelet agglutination took place immediately, and after one minute, the relative agglutination ratio increased to about 45%.

### EXAMPLE 7

In this example, hepatitis inhibition action of methanol extracts of ume kernel and ume leaf-stem was confirmed. The methanol extracts were prepared in the same manner as in Example 1.

Confirmation of D-galactosamine/LPS Induced Acute Hepatitis Inhibition

To ddY male mice (10 mice, 25 to 30 g in weight) fasted for about 20 hours, the ume extract was orally administered at a ratio of 1000 mg/kg. After one hour, D-galactosamine and lipopolysaccharide (LPS) were intraperitoneally administered at a ratio of 350 mg/kg and a ratio of 10 μg/kg, respectively. After 10 hours, blood was drawn, and a serum was obtained by centrifugation (3000 rpm, 10 minutes, 4° C.). The activities of transaminases (s-GPT, s-GOT) in the serum were measured using a commercial kit (S-TA TEST WACO, manufactured by Wako Pure Chemical Industries, Ltd.). Furthermore, 10 mice were subjected to the same treatment except that the ume extract was not administered, and they were used as a control group. Then, the ratio of the transaminases activity of the mice treated with the ume extract with respect to the control group was calculated as an inhibition ratio (%). This result is shown in Table 5 below.

TABLE 5

| | s-GPT | s-GOT |
|---|---|---|
| Inhibition Ratio (%) | 26.6 ± 15.8 | 32.6 ± 11.7 |

Action of Inhibiting D-galactosamine Induced Acute Hepatitis in Primary Culture Hepatic Cells of Rat

Hepatocytes obtained from Wistar male rats (120 to 150 g in weight) by a collagenase perfusion method were suspended in a William's medium containing 10% calf serum (CG). 4×10⁴ cells/ml of the suspension was seeded in a 96-well flat-bottom microplate, and incubated for 4 hours. Subsequently, the medium was exchanged with a medium containing 1 mM D-galactosamine and the ume extract (concentration: 3, 10, 30, 100, 300 ttμg/ml), and incubated for 44 hours. Thereafter, 10 gl of 5 mg/ml 3-(4,5-dimethylthiazol-2-yl)-2,5-diphenyltetrazolium bromide (MTT) was added to each well, and incubated for 4 hours. Then, the medium was removed, and a formazan was extracted with 2-propanol containing 0.04 N HCl. Absorbance of the formazan was measured (measurement wavelength: 570 nm, reference wavelength: 655 nm). Furthermore, a sample subjected to the same treatment except that the ume extract was not added to the medium was used as a control. The ratio of the measured value when the ume extract was added with respect to the control was calculated as an inhibition ratio (%). This result is shown in Table 6 below.

TABLE 6

| | Extract Concentration | | | | |
|---|---|---|---|---|---|
| | (μg/ml) | | | | |
| | 3 | 10 | 30 | 100 | 300 |
| Inhibition | 5.2 ± 0.5 | 9.4 ± 0.8 | 15.4 ± 1.0 | 7.9 ± 0.7 | 2.8 ± 0.2 |
| Ratio (%) | | | | | |

As is apparent from the Tables 5 and 6, acute hepatitis was inhibited by administering the ume extract.

### EXAMPLE 8

In this example, inflammation inhibition action of a methanol extract of ume leaf-stem was confirmed. The methanol extract of ume leaf-stem was prepared in the same manner as in Example 1.

Confirmation of Inflammation Inhibition Action

Inflammation inhibition action was evaluated by investigating the NO production from macrophages in the abdominal cavity of a mouse due to LPS (10 μg/ml) stimulation.

Measurement of NO From Macrophages

Macrophages obtained from the abdominal cavity of a ddY male mouse (about 30 g in weight) were suspended in an RPMI-1640 medium containing 10% calf serum (FCS). 5×10⁵ cells/ml of the suspension was seeded in a 96-well flat-bottom microplate, and incubated for 1 hour (37° C., 5% CO₂). Subsequently, the medium was exchanged with a medium containing 10 μg/ml of LPS and the ume extract (concentration: 3, 10, 30, 100, 300 μg/ml), and incubated for 20 hours. Because NO is unstable and is difficult to measure directly, NO₂ as a metabolic product of NO was quantitatively determined using a Griess reagent (Griess method). That is, a supernatant of the culture and the same amount of a Griess reagent (1% sulfanilamide/0.1% N-1-naphthylethylenediamine/5% phosphoric acid) were mixed, and allowed to stand at room temperature for 10 minutes. Thereafter, the absorbance was determined (measurement wavelength: 570 nm, reference wavelength: 655 nm), and NO₂ was quantitatively determined using NaNO₂ diluted with the medium as a standard. A sample subjected to the same treatment except that the ume extract was not added was used as a control. The ratio of the measured value when the ume extract was added with respect to the control was calculated as an inhibition ratio (%). This result is shown in Table 7 below.

TABLE 7

| | Extract Concentration | | | | |
|---|---|---|---|---|---|
| | (μg/ml) | | | | |
| | 3 | 10 | 30 | 100 | 300 |
| Inhibition | 26.8 ± 2.2 | 2.7 ± 1.7 | 5.7 ± 1.6 | 13.5 ± 1.4 | 30.5 ± 0.9 |
| Ratio (%) | | | | | |

As is apparent from the Table 7, inflammation was inhibited by administering the ume extract.

### EXAMPLE 9

In this example, the action of inhibiting melanin pigment formation of a methanol extract of ume leaf-stem was confirmed. The methanol extract of ume leaf-stem was prepared in the same manner as in Example 1.

Confirmation of the Action of Inhibiting Melanin Pigment Formation

Evaluation was made according to the inhibition activity with respect to a mushroom-derived tyrosinase. That is, 0.1 ml of 0.25 mg/ml tyrosinase (derived from mushroom) was added to a mixture solution (1.9 ml) of 40 mM phosphate buffer solution (pH 6.8) containing the ume extract (concentration: 30, 100, 300 μg/ml) and a substrate (L-dopa), and incubated at 25° C. for 5 minutes. Thereafter, the absorbance was determined at 475 nm. Furthermore, a sample subjected to the same treatment except that the ume extract was not added was used as a control. The ratio of the measured value when the ume extract was added with respect to the control was calculated as an inhibition ratio (%). This result is shown in Table 8 below.

TABLE 8

| | Extract Concentration | | |
|---|---|---|---|
| | (μg/ml) | | |
| | 30 | 100 | 300 |
| Inhibition | 3.4 | 10.5 | 34.3 |
| Ratio (%) | | | |

As is apparent from the Table 8, melanin pigment formation was inhibited by administering the umne extract.

### EXAMPLE 10

In this example, substances assumed to contribute to the above-mentioned various medicinal effects were purified.

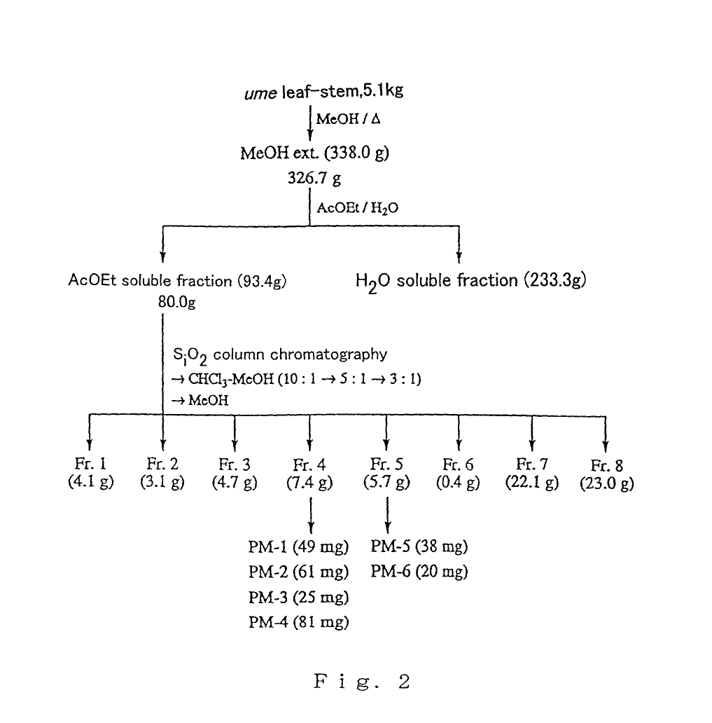

First, in the same manner as in Example 1, a methanol extract (338.0 g) of ume leaf-stem was prepared. 326.7 g of this extract was divided into an AcOEt soluble fraction (93.4 g) and a water soluble fraction (233.3 g) using 10 liters of ethyl acetate (AcOEt) and 10 liters of distilled water (H₂O). Then, using silica column chromatography, the AcOEt soluble fraction (80.0 g) was divided into eight fractions (Fr. 1 to Fr. 8). In this fractioning, first a mixture solution of hexane (Hex)—AcOEt (volume ratio; 10:1→5:1→3:1) was used, then a mixture solution of CHCl₃— methanol (MeOH) (volume ratio; 10:1→5:1→3:1) was used, and finally a MeOH solution was used. As a result, in the fraction of Fr. 4 (CHCl₃:MEOH=10:1), PM-1 (49 mg), PM-2 (61 mg), PM-3 (25 mg) and PM-4 (81 mg) were obtained. In the fraction of Fr. 5 (CHCl₃:MEOH=5:1), PM-5 (38 mg) and PM-6 (20 mg) were obtained. The purification process of these substances is shown in FIG. 2.

### EXAMPLE 11

In this example, the activity of inhibiting a rat lens-derived aldose reductase of a methanol extract of ume flower was confirmed. Aldose reductase is involved in diabetic cataract and diabetic neurosis. The methanol extract of ume flower was prepared as described below. Using this extract, aldose reductase inhibition activity was investigated as described below.

Preparation of Methanol Extract of Ume Flower

3.0 kg of finely ground ume flower was heat refluxed for 3 hours with methanol in a volume of five times the volume of the ume flower to carry out extraction. The resultant extract was filtered, and thereafter the residue was heat refluxed again with methanol in the same manner as the above to carry out extraction. The obtained extracts were joined, and dried under reduced pressure to obtain 250.1 g of an extract. The properties of this extract are shown below.

Appearances and Properties

Dark brown, powdery, with a trace of peculiar odor.

Thin Layer Chromatography

(1) Conditions

Carrier: Silica gel (60F254, manufactured by Merck & Co., Inc.)

Developing Solvent: A mixture solution of chloroform: methanol: water=10:3:1 (volume ratio)

(2) Rf value

Spot 1: 0.70 (yellow)

Spot 2: 0.63 (yellow)

Preparation of Aldose Reductase Solution

5 g of lens of Wistar male rats (6-week old) was homogenized in 20 ml of a phosphate buffer solution (135 mM, pH 7.0, containing 10 mM mercaptoethanol), and centrifuged at 100,000×g for 30 minutes. The resultant supernatant was used as an enzyme solution.

Confirmation of Aldose Reductase Inhihition Activity

1 mM DL-glyceraldehyde, 0.03 mM NADPH, 0.1 M lithium sulfate, the rat lens-derived aldose reductase solution, a DMSO solution of the extract (concentration: 1, 3, 10, 30 μg/ml), and 135 mM phosphate buffer solution (pH 7.0) were mixed, and the mixture was incubated at 30° C. for 30 minutes. The reaction was initiated by adding NADPH, and quenched by adding hydrochloric acid. The quenched reaction solution was treated with a strong alkali, and thereafter heated at 60° C. for 10 minutes. Thereafter, the formed NADP was quantitatively determined by a fluorescence method (excitation wavelength: 360 nm, radiation wavelength: 460 nm). The obtained value was substituted in the equation below to calculate the ratio of aldose reductase inhibition. This result is shown in Table 9 below.

Inhibition ratio (%)=[(A-B)/A]×100

A: The amount of NADP formed when the extract was not added

B: The amount of NADP formed when the extract was added

TABLE 9

Extract Concentration (μg/ml)

| | 1 | 3 | 10 | 30 |
|---|---|---|---|---|
| Inhibition Ratio (%) | 29.9 | 51.2 | 76.8 | 89.6 |

As is apparent from the Table 9, the methanol extract of ume flower had excellent aldose reductase inhibition activity (IC₅₀=3 μg/ml).

### EXAMPLE 12

In this example, inflammation inhibition action of a methanol extract of ume flower was confirmed. The methanol extract of ume flower was prepared in the same manner as in Example 11.

Confirmation of Inflammation Inhihition Action

Inflammation inhibition action was evaluated by investigating the NO production from macrophages in the abdominal cavity of a mouse due to LPS (10 μg/ml) stimulation.

Measurement of NO From Macrophages

Macrophages obtained from the abdominal cavity of a ddY male mouse (about 30 g in weight) were suspended in an RPMI-1640 medium containing 10% calf serum (FCS). 5×10⁵ cells/ml of the suspension was seeded in a 96-well microplate, and incubated for 1 hour (37° C., 5% CO₂). Subsequently, the medium was exchanged with a medium containing 10 μg/ml of LPS and the ume extract (concentration: 3, 10, 30, 100, 300 μg/ml), and incubated for 20 hours. Because NO is unstable and is difficult to measure directly, NO₂ as a metabolic product of NO was quantitatively determined using a Griess reagent (Griess method). That is, a supernatant of the culture and the same amount of a Griess reagent (1% sulfanilamide/0.1% N-1-naphthylethylenediamine/5% phosphoric acid) were mixed, and the mixture was allowed to stand at room temperature for 10 minutes. Thereafter, the absorbance was determined (measurement wavelength: 570 nm, reference wavelength: 655 nm), and NO₂ was quantitatively determined using NaNO₂ diluted with the medium as a standard. A sample subjected to the same treatment except that the ume extract was not added was used as a control. The ratio of the measured value when the ume extract was added with respect to the control was calculated as an inhibition ratio (%). This result is shown in Table 10 below.

TABLE 10

| | Extract Concentration | | | | |
|---|---|---|---|---|---|
| | (μg/ml) | | | | |
| | 3 | 10 | 30 | 100 | 300 |
| Inhibition | 8.0 ± | 1.7 ± 4.8 | 5.0 ± 3.8 | 51.6 ± 6.0* | 94.8 ± 1.2* |
| Ratio (%) | 4.5 | | | | |

As is apparent from the Table 10, the methanol extract of ume flower exhibited excellent action of inflammation inhibition (IC₅₀=100 μg/ml).

### EXAMPLE 13

In this example, the action of inhibiting melanin pigment formation of a methanol extract of ume flower was confirmed. The methanol extract of ume flower was prepared in the same manner as in Example 11.

Confirmation of the Action of Inhihiting Melanin Pigment Formation

Evaluation was made based on the inhibition activity with respect to a mushroom-derived tyrosinase. That is, a 40 mM phosphate buffer solution (pH 6.8) containing a DMOS solution of the ume extract (concentration: 1, 3, 10, 30, 100, 300, 1000 μg/ml), 0.1 mg/ml of L-dopa and 500 U/ml of a tyrosinase (derived from mushroom) was incubated at 25° C. for 5 minutes. Thereafter, the absorbance was determined at 475 nm. Furthermore, a sample subjected to the same treatment except that the ume extract was not added was used as a control. The ratio of the measured value when the ume extract was added with respect to the control was calculated as an inhibition ratio (%). This result is shown in Table 11 below.

TABLE 11

| | Extract Concentration | | | | | | |
|---|---|---|---|---|---|---|---|
| | (μg/ml) | | | | | | |
| | 1 | 3 | 10 | 30 | 100 | 300 | 1000 |
| Inhibition | 2.3 | 7.5 | 11.3 | 24.1 | 46.3 | 63.5 | 77.5 |
| Ratio (%) | | | | | | | |

As is apparent from the Table 11, melanin pigment formation was inhibited by administering the ume extract.

### EXAMPLE 14

In this example, anti-oxidation action of a methanol extract of ume flower was confirmed. The methanol extract of ume flower was prepared in the same manner as in Example 11.

Method of Confirming Anti-oxidation Action

1.0 ml of 0.1 M acetic acid-sodium acetate buffer solution (pH 5.5), 0.5 ml of ethanol, 0.5 ml of 2.0×10⁻⁴ M DPPH ethanol solution, and 0.5 ml of an ethanol solution of the methanol extract of ume flower were mixed, and the mixture was allowed to stand at room temperature for 30 minutes. Thereafter, absorbance was determined at 517 nm. From the obtained value, the amount of the extract necessary to reduce 2.0×10⁻⁷ M DPPH radicals by 50% was calculated. As a result, the amount of the extract necessary to reduce the DPPH radicals by 50% was 44 μg. This result shows that the ume flower extract had excellent anti-oxidation activity.

### EXAMPLE 15

In this example, the action of inhibiting platelet agglutination of a methanol extract of ume flower was confirmed. The methanol extract of ume flower was prepared in the same manner as in Example 11.

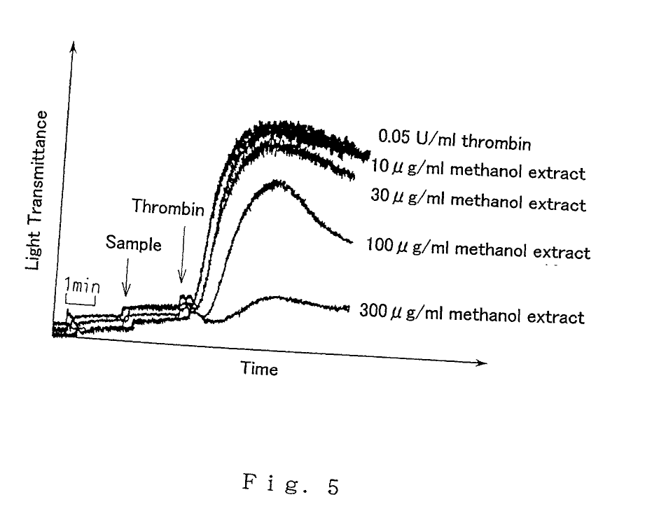

First, whole blood was drawn from the auricular artery of a Japan white male rabbit, and then it was centrifuged to obtain a multiple platelet plasma. It was washed with a Tyrode-Hepes solution containing 0.4 mM EGTA to prepare washed platelets (5×10⁵ cells/ml). Then, in the presence of the ethanol extract of ume flower, the washed platelets were stimulated with 0.05 U/ml of thrombin for 5 minutes, and the change in light transmittance at this time was measured with the above-mentioned aggregometer. The measured value was substituted in the following equation to calculate a platelet agglutination inhibition ratio (%). This result is shown in Table 12 below and in the chart of FIG. 5.

Inhibition ratio (%) [(A-B)/A]×100

A: Maximum agglutination ratio when the extract was not added

B: Maximum agglutination ratio when the extract was added

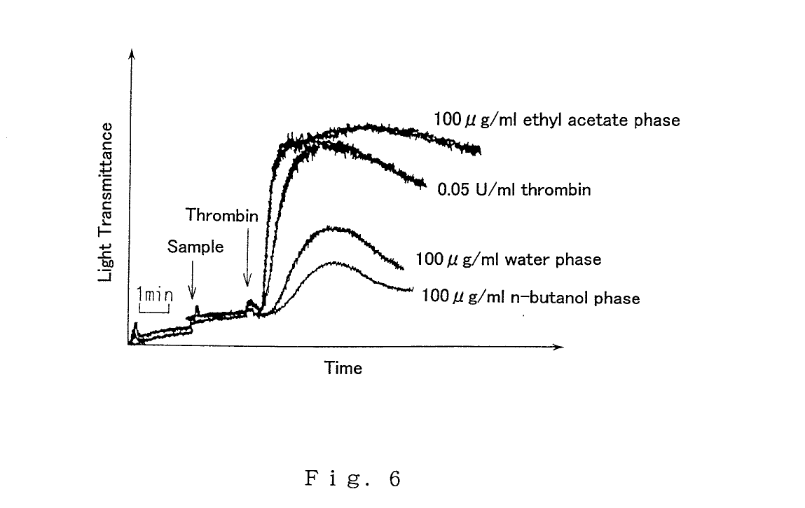

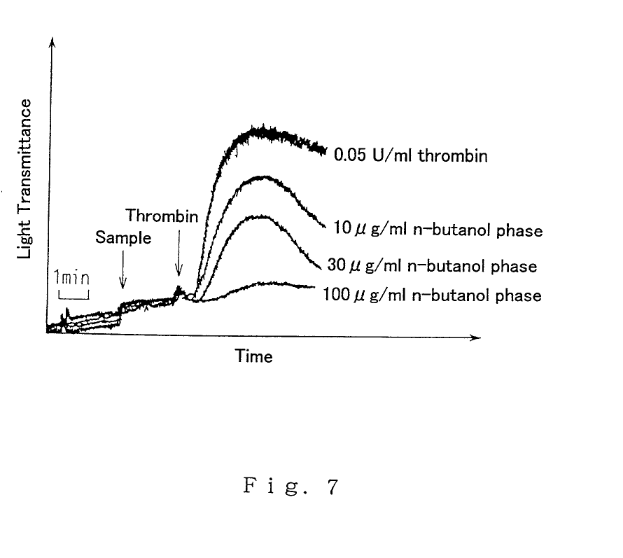

Furthermore, the methanol extract of ume flower was divided with ethyl acetate and water, and the action of inhibiting platelet agglutination in each of the ethyl acetate phase and the water phase was investigated by the above-mentioned method. This result is shown in Table 12 below and in the chart of FIG. 6. Then, the water phase was shifted further to n-butanol, and the action of inhibiting platelet agglutination in the n-butanol phase was investigated by the above-mentioned method. This result is shown in Table 12 below and in the chart of FIG. 7. In FIGS. 5, 6 and 7, the “sample” indicates the point when the extract or each phase was added, and the “thrombin” indicates the point when thrombin was added.

TABLE 12

| | Concentration (μg/ml) | | |
|---|---|---|---|
| | 10 | 30 | 300 |
| (Inhibition ratio (%)) | | | |
| Methanol extract | 0 | 10 | 28 |
| Ethyl acetate phase | — | — | 5 |
| Water phase | 11 | 27 | 89 |
| n-butanol phase | 96 | 87 | 96 |

As is apparent from the Table 12 and the charts of FIGS. 5, 6 and 7, the action of inhibiting platelet agglutination of the methanol extract of ume flower was confirmed. When this methanol extract was divided with ethyl acetate and water, although platelet agglutination inhibition activity was not confirmed in the ethyl acetate phase, remarkable activity of inhibiting platelet agglutination was confirmed in the water phase. Furthermore, when the water phase was extracted with n-butanol, intensive activity was observed in the n-butanol phase. On the other hand, the methanol extract of ume flower hardly inhibited the platelet agglutination due to a platelet activation factor (PAF) (not illustrated). According to the above results, it was confirmed that an extract of ume flower specifically inhibits thrombin agglutination. According to these results, it is assumed that an extract of ume flower has effects of prevention and improvement with respect to diseases caused by thrombosis, such as cardiac infarction and brain infarction.

### EXAMPLE 16

In this example, the above-mentioned various substances in a methanol extract of ume flower were purified.

Preparation of Methanol Extract of Ume Flower

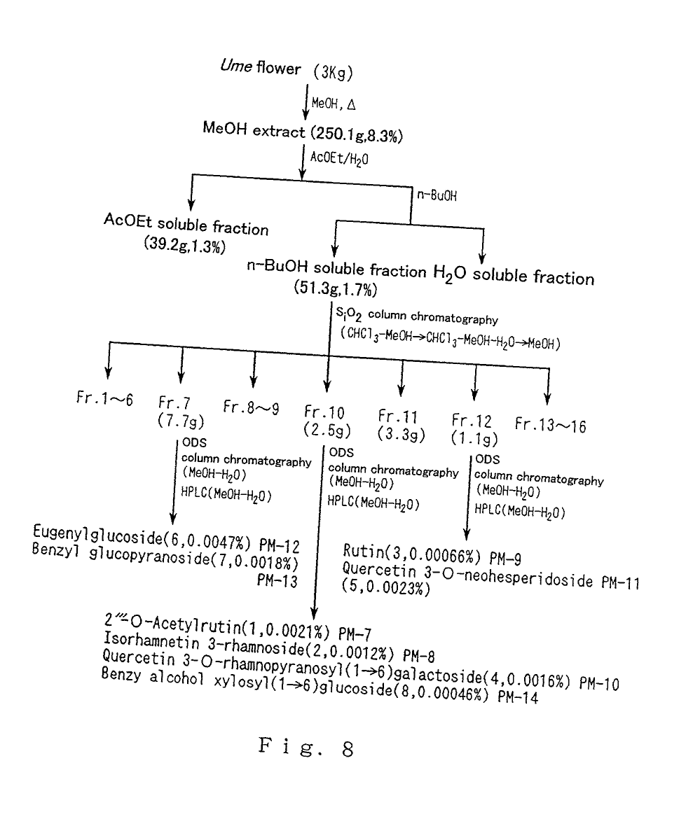

3.0 kg of finely ground ume flower was heat refluxed for 3 hours with methanol in a volume of five times that of the ume flower to carry out extraction. The extract was filtered, and thereafter the residue was heat refluxed again with methanol in the same manner as the above to carry out extraction, and the extract was filtered. Then, the obtained extracts were joined, and dried under reduced pressure to prepare 250.1 g of a methanol extract of ume flower. Then, 250.1 g of this extract was divided into an AcOEt soluble fraction (39.2 g), a BuOH soluble fraction (51.2 g) and a water soluble fraction (150.0 g), using 10 liters of ethyl acetate (AcOEt), 10 liters of butanol (BuOH) and 10 liters of distilled water (H₂O). Then, the BuOH soluble fraction (25.1 g) was divided into 16 fractions (Fr. 1 to Fr. 16) using normal phase silica column chromatography. In this fractioning, first, a mixture solution of chloroform (CHCl₃)—MeOH (volume ratio; 10:1→5:1) was used, then a mixture solution of CHCl₃—MeOH—H₂O (volume ratio; 6:4:1) was used, and finally a MeOH solution was used. As a result, PM-12 (eugenylglucoside: 141 mg) and PM-13 (benzyl glucopyranoside: 54 mg) were obtained in the fraction of Fr. 7 (CHCl₃:MeOH=5:1); PM-7 (2′″-O-acetylrutin: 63 mg), PM-8 (isorhamnetin: 36 mg), PM-10 (quercetin 3-O-rhamnopyranosyl (1→6) galactoside: 48 mg) and PM-14 (benzyl alcohol xylosyl (1→6) glucoside: 14 mg) were obtained in the fraction of Fr. 10 (CHCl₃:MeOH:H₂O=6:4:1); and PM-9 (rutin: 20 mg) and PM-11 (quercetin 3-O-neohesperidoside: 69 mg) were obtained in the fraction of Fr. 12 (CHCl₃:MeOH:H₂O=6:4:1). The purification process of these substances is shown in FIG. 8.

Industrial Applicability

As described above, the present invention provides ume extracts having the above-mentioned various medicinal effects. Thus, while the present invention can contribute to effective use of ume, it can also provide medicines useful for diseases that have become social problems.

1. An ume extract having a medicinal effect, the ume extract being extracted from at least one selected from the group consisting of a main stem of ume tree, a branch of ume tree, a leaf of ume tree, a stem of ume tree, a root of ume tree, ume flesh, seed and shell of ume, and a kernel of ume, wherein:
the ume extract is used as an antioxidant.

2. An ume extract having a medicinal effect, the ume extract being extracted from at least one selected from the group consisting of a main stem of ume tree, a branch of ume tree, a leaf of ume tree, a stem of ume tree, a root of ume tree, ume flesh, seed and shell of ume, and a kernel of ume, wherein:
the ume extract is used as a gastric mucosa injury inhibitor.

3. (Amended) An ume extract having a medicinal effect, the ume extract being extracted from at least one selected from the group consisting of a main stem of ume tree, a branch of ume tree, a leaf of ume tree, a stem of ume tree, a root of ume tree, ume flesh, seed and shell of ume, and a kernel of ume, wherein:
the ume extract has aldose reductase inhibition activity, and is used as a diabetic cataract preventing agent with the activity.

4. (Amended) An ume extract having a medicinal effect, the ume extract being extracted from at least one selected from the group consisting of a main stem of ume tree, a branch of ume tree, a leaf of ume tree, a stem of ume tree, a root of ume tree, ume flesh, seed and shell of ume, and a kernel of ume, wherein:
the ume extract has aldose reductase inhibition activity, and is used as a diabetic neurosis preventing agent with the activity.

5. An ume extract having a medicinal effect, the ume extract being extracted from at least one selected from the group consisting of a main stem of ume tree, a branch of ume tree, a leaf of ume tree, a stem of ume tree, a root of ume tree, ume flesh, seed and shell of ume, and a kernel of ume, wherein:
the ume extract is used as a blood glucose level elevation inhibitor.

6. An ume extract having a medicinal effect, the ume extract being extracted from at least one selected from the group consisting of a main stem of ume tree, a branch of ume tree, a leaf of ume tree, a stem of ume tree, a root of ume tree, ume flesh, seed and shell of ume, and a kernel of ume, wherein:
the ume extract is used as an alcohol absorption inhibitor.

7. An ume extract having a medicinal effect, the ume extract being extracted from at least one selected from the group consisting of a main stem of ume tree, a branch of ume tree, a leaf of ume tree, a stem of ume tree, a root of ume tree, ume flesh, seed and shell of ume, and a kernel of ume, wherein:
the ume extract is used as a platelet agglutination accelerator.

8. An ume extract having a medicinal effect, the ume extract being extracted from at least one selected from the group consisting of a main stem of ume tree, a branch of ume tree, a leaf of ume tree, a stem of ume tree, a root of ume tree, ume flesh, seed and shell of ume, and a kernel of ume, wherein:
the ume extract is used as a hepatitis inhibitor.

9. An ume extract having a medicinal effect, the ume extract being extracted from at least one selected from the group consisting of a main stem of ume tree, a branch of ume tree, a leaf of ume tree, a stem of ume tree, a root of ume tree, ume flesh, seed and shell of ume, and a kernel of ume, wherein:
the ume extract is used as an anti-inflammatory agent.

10. (Amended) An ume extract having a medicinal effect, the ume extract being extracted from at least one selected from the group consisting of a main stem of ume tree, a branch of ume tree, a leaf of ume tree, a stem of ume tree, a root of ume tree, ume flesh, seed and shell of ume, and a kernel of ume, wherein:
the ume extract has tyrosinase inhibition activity, and is used as a melanin pigment formation inhibitor with the activity.

11. An ume extract according to any one of claims 1, 3, 4, 5, 7, 8, 9 and 10, wherein the ume extract is extracted from at least one of a leaf of ume tree and a stem of ume tree.

12. An ume extract according to any one of claims 2, 5, 6 and 8, wherein the ume extract is extracted from a kernel of ume.

13. An ume extract according to any one of claims 1 to 10, wherein the ume extract comprises at least one substance selected from the group consisting of six substances represented by chemical formulae (1) to (6) below:

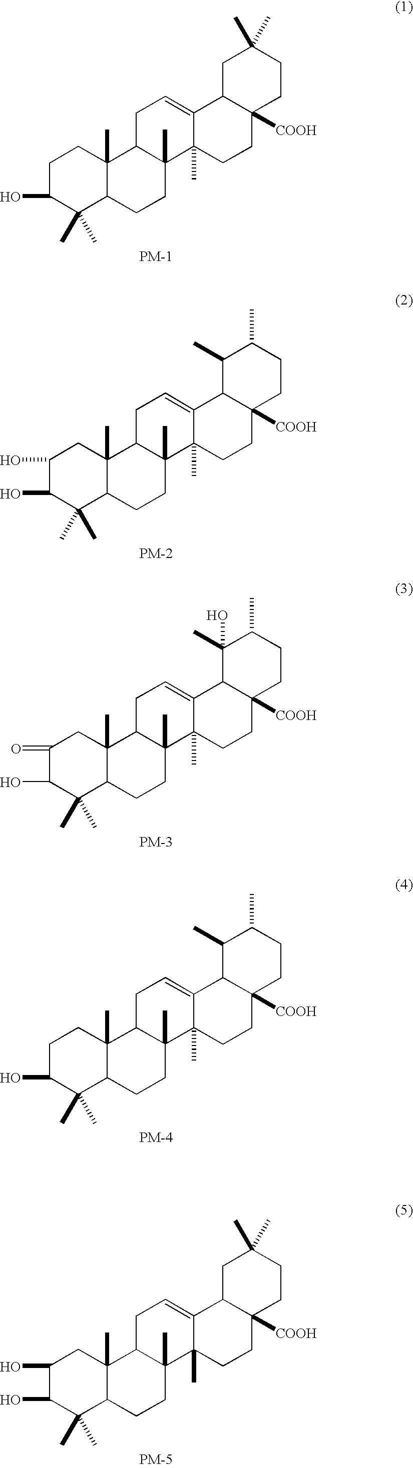

14. (Amended) An ume extract having a medicinal effect, the ume extract being extracted from an ume flower, wherein:
the ume extract has aldose reductase inhibition activity, and is used as a diabetic cataract preventing agent with the activity.

15. (Amended) An ume extract having a medicinal effect, the ume extract being extracted from an ume flower, wherein:
the ume extract has aldose reductase inhibition activity, and is used as a diabetic neurosis preventing agent with the activity.

16. An ume extract having a medicinal effect, the ume extract being extracted from an ume flower, wherein:
the ume extract is used as an anti-inflammatory agent.

17. An ume extract having a medicinal effect, the ume extract being extracted from an ume flower, wherein:
the ume extract is used as an antioxidant.

18. (Amended) An ume extract having a medicinal effect, the ume extract being extracted from an ume flower, wherein:
the ume extract has tyrosinase inhibition activity, and is used as a melanin pigment formation inhibitor with the activity.

19. An ume extract having a medicinal effect, the ume extract being extracted from an ume flower, wherein:
the ume extract is used as a platelet agglutination inhibitor.

20. An ume extract according to any one of claims 14 to 19, wherein the ume extract comprises at least one substance selected from the group consisting of eight substances represented by chemical formulae (7) to (14) below:

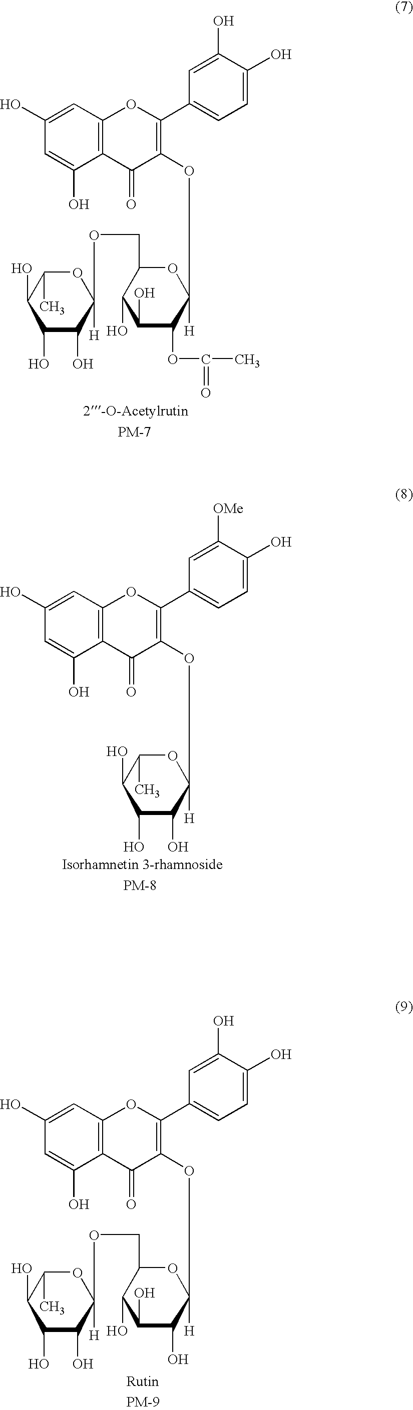

21. An ume extract according to any one of claims 1 to 10 and claims 14 to 19, wherein the ume extract is an organic solvent extract.

22. An ume extract according to claim 21, wherein the organic solvent extract is an alcohol extract.

23. An ume extract according to claim 22, wherein the alcohol extract is at least one of an ethanol extract and a methanol extract.

24. An ume extract according to claim 22, wherein the alcohol extract is a methanol extract.

25. An ume extract according to any one of claims 1 to 10 and claims 14 to 19, wherein the ume extract is a dry distillation extract.

26. An ume extract according to any one of claims 1 to 10 and claims 14 to 19, wherein a form of the ume extract is powder.

27. An ume extract according to any one of claims 1 to 10 and claims 14 to 19, wherein a form of the ume extract is liquid.

28. A composition comprising an ume extract according to any one of claims 1 to 10 and claims 14 to 19.
